(19) United States
(12) Patent Application Publication
(10) Pub. No.: US 2025/0384454 A1
(43) Pub. Date: Dec. 18, 2025

(54) METHOD AND SYSTEM FOR PHARMACEUTICAL PORTFOLIO STRATEGIC MANAGEMENT DECISION SUPPORT BASED ON ARTIFICIAL INTELLIGENCE

(71) Applicant: GROUPE SORINTELLIS INC., Montréal (CA)

(72) Inventors: Emmanuel Piffo, Laval, QC (CA); Diana Avramioti, Laval, QC (CA); Jordan Gierschendorf, Montreal, QC (CA); Camelia Raymond, Montreal, QC (CA)

(21) Appl. No.: 18/878,329

(22) PCT Filed: Jun. 23, 2023
(86) PCT No.: PCT/CA2023/050878

§ 371 Date: Dec. 23, 2024

Related U.S. Application Data
(60) Provisional application No. 63/366,875, filed on Jun. 23, 2022.

(51) Int. Cl. G06Q 30/0202; G16H 10/20; G16H 20/10; G16H 50/20

(52) U.S. Cl. CPC G06Q 30/0202; G16H 10/20; G16H 20/10; G16H 50/20

(57) ABSTRACT

This invention is a method, system, and platform for risk management and decision support in pharmaceuticals, including strategic portfolio management, regulatory affairs, clinical drug development, pharmacoeconomic, investment strategy optimization, risk management, due diligence for mergers and acquisitions, and the stock market. The system uses artificial intelligence and diverse data from open and private sources, including clinical trials, regulatory decisions, economic data, pharmacological data, and corporate data. It integrates multiple decision-making modules for clinical development, such as clinical risk, regulatory risk, pharmacological risk, and economic risk. This network of risk factors generates predictive and prescriptive information for strategic decision-making.

### CROSS-REFERENCE TO RELATED APPLICATIONS

The present patent application claims the benefits of priority of U.S. Provisional Patent Application No. 63/366,875, entitled “METHOD AND SYSTEM FOR PHARMACEUTICAL PORTFOLIO STRATEGIC MANAGEMENT DECISION SUPPORT BASED ON ARTIFICIAL INTELLIGENCE” and filed at the United States Patent and Trademark Office on Jun. 23, 2022, the content of which is incorporated herein by reference.

### FIELD OF THE INVENTION

The present invention generally relates to methods and systems for pharmaceutical portfolio strategic management decision support based on artificial intelligence. More specifically, the present invention relates to platforms and portals using methods and systems for pharmaceutical portfolio strategic management decision support based on artificial intelligence.

### BACKGROUND OF THE INVENTION

The drug development process is well known to be a lengthy, costly, complex, and above all a very high-risk endeavor. When successful drug development is achieved, colossal revenue generation can be expected for the pharmaceutical companies and the life sciences ventures funds. However, this process is plagued with some major challenges. Indeed, pharmaceutical companies spend more than a decade and often exceed $2 billion for bringing a new therapeutic product from laboratory to market. Additionally, the cost of developing a new drug as well as the total R&D expenditure have increased, while the rate of introduction of a new product on the market has remained approximately constant over time, resulting in what some authors qualified as the pharmaceutical R&D productivity crisis (Pammolli et al., 2011). Given the persistent productivity crisis in the pharmaceutical industry, we are in position to question the sustainability of the biopharmaceutical industry's business.

The global innovative pharmaceutical industry, whose core business remains pharmaceutical innovation, is therefore facing major challenges and is multiplying strategies to reduce development risks and increase the productivity of its pipeline. One of the major milestones in drug development projects, if not the most important, is obtaining regulatory approval for marketing authorization from regulatory agencies (e.g., US FDA, Health Canada).

Knowing that successfully advancing the clinical trial phases is necessary to finally obtain regulatory approval, the success of the clinical trial phases therefore becomes a critical step. A Phase I study is intended to determine the safety and tolerability of a drug candidate in humans. It is traditionally conducted in healthy participants but could be conducted in patients in contexts where it is ethically inadmissible to do so in healthy volunteers. A Phase II study is planned to test the efficacy and safety of a drug candidate in a larger group of patients with the disease or condition for which the treatment is intended. A Phase III study, called a pivotal study (the most expensive clinical phase), must confirm the efficacy and safety of a drug candidate in a large group of patients with the disease. It is estimated that approximately one on ten potential medicines that start phase I clinical trials will enter the market (Hay et al., 2014). Indeed, between 60% to 70% of phase II will not progress to phase III and 30% to 40% of phase III will not progress to market. Moreover, only about 20% of New Molecular Entity (NME) cover their average capitalized R&D expenses and not all marketed drugs will generate revenues that match or exceed R&D costs (Vernon et al., 2010). This means that obtaining regulatory approval is no guarantee of a drug's commercial success. These statistics show the significant challenges for both pharmaceutical companies, contract research organizations (CRO) and life science venture capital (LSVC).

Risk is inherent to this industry whose fundamental vocation is pharmaceutical innovation. However, the nature of risk in this industry is multidimensional. One important dimension involves clinical development since late-stage development failures are the costliest. For every new drug launched, clinical development (Phases I-III) accounts for approximately 63% of the costs and preclinical drug discovery accounts for only 32%. Indeed, if pharmacological efficacy and safety remain well-known failure factors, the literature abounds on a multitude of important failure risk factors of a strategic, operational, and commercial nature. In addition to being difficult for humans to control, the decisions associated with these risk factors are major contributors to failure in clinical development (Phase I, II, III).

Solving this problem requires the development and implementation of effective portfolio management strategies in the pharmaceutical industry. While in areas such as finances, portfolio risk management strategies are highly developed, the pharmaceutical industry has been slow to adapt effective approaches to effectively manage its pipeline. An effective and efficient portfolio management strategy in this industry cannot be deployed without intelligent a priori and real-time risk monitoring throughout the drug development process. Artificial Intelligence (AI) gives us the opportunity to revolutionize these crucial steps in drug development by enabling intelligent control of the multitude of avoidable strategic and operational risk factors for failure.

### Pharmaceutical Pipeline Productivity

Pharmaceutical pipeline productivity can be defined as the ratio of the value (therapeutic and commercial) created by a new drug to the investment required to generate that drug (Paul et al., 2010a). Studies point to the productivity crisis in the pharmaceutical pipeline. Indeed, data show that during the last decades the number of approved new molecules has decreased significantly while total R&D expenditures have increased considerably (Pammolli et al., 2011). The repeated observation of this phenomenon in the pharmaceutical industry has even raised concerns for many authors about the sustainability of the biopharmaceutical industry's business model (Paul et al., 2010b; Scannell et al., 2012; Schlander et al., 2021). Therefore, it has become imperative over the years for the pharmaceutical industry to find new approaches to optimize strategic decision making in pharmaceutical portfolio/pipeline management. Indeed, like any other organization, the pharmaceutical industry is constantly looking for efficiency in its decision-making processes to optimize the use of its resources and increase its ROI. Then the productivity crisis as defined here represents a major challenge for pharmaceutical portfolio managers.

### Drug Development Process

Substantially, the drug development process can be segmented technically and temporally as a sequence of two major stages, namely the Drug Discovery (Preclinical studies) and the Drug Development (Clinical studies in humans).

Drug Discovery is the initial selection of the most promising molecules as drug candidates that will enter Phase I (first study in humans). Drug Development corresponds to the stages of clinical development, which essentially include interventional clinical studies conducted in humans. They include Phase I, II and III studies.

Fundamentally, Phase I trials often referred to as first-in-human studies, are specifically concerned with evaluating the safety of the drug candidate in a dose-dependent manner. The tests are usually done on a small group of healthy volunteers. However, it is possible to conduct Phase I studies on patients when it is not ethically appropriate to conduct them on healthy volunteers.

Basically, Phase II studies are used to evaluate the short-term efficacy and toxicity of the drug candidate. These studies are generally used to establish proof of concept (Proof of concept studies). These studies are conducted on hundreds of patients, suffering from the therapeutic conditions of interest. The scientific robustness (elimination of bias) of these studies very often requires that the design of these studies is usually randomized controlled single or double blind. One group receives the experimental drug while the second group, called the control group, is given a comparator drug or placebo (if ethically acceptable).

Blinded studies mean that the patient does not know what treatment is being administered. In double-blind studies, neither the patient nor the clinical researchers know who is receiving the experimental treatment. There are even triple-blind studies where in addition to the patient and the clinical investigator, the statistical investigator who analyzes the data are all kept blind to the randomization data.

Phase III or pivotal studies evaluate the efficacy and safety of the drug candidate on patients suffering from the medical condition of interest. They are generally randomized and controlled trials to ensure the robustness of the data collected. This is the pivotal step before a marketing application is submitted to the marketing regulatory agencies (e.g., Health Canada).

While it is estimated that it takes approximately $2 billion to develop a drug, i.e., to conduct the discovery and development phases as defined above, it is important to remember that it is the late clinical development phases that are the costliest for the pharmaceutical industry. Despite numerous technological advances to improve the chances of success in the discovery phase, attrition rates in clinical development remain high. However, it is late-stage failures that are the costliest compared to early-stage discovery. Indeed, for every new drug launched, clinical development (Phases I-III) accounts for approximately 63% of costs and preclinical drug discovery accounts for only 32%.

Phase transitions in clinical development are defined as moving from one phase to the next, (e.g., from Phase II to Phase III). The historical data compiled allows us to observe statistically that the probability of transition from one phase to the next is variable and depends on many factors such as therapeutic area, type of molecule etc.

According to the US Food and Drug Administration, 30% of compounds fail in phase I, 67% fail in phase II and between 67% and 75% of compounds will never be marketed despite having reached the final stages of clinical development. Other published work has reported success rates of 51% for discovery phases, 69% for preclinical development phases, 12.8% for clinical development phases (Phases I, II, and II), which translates into an overall probability of technical and regulatory success of 4.1% (Paul et al., 2010). According to The Pharmaceutical Research and Manufacturers of America (PhRMA), the overall industry success rate would be hands down 12%. In other words, this would mean that only one in ten molecules that enter clinical development is ultimately approved.

Pharmaceutical portfolio managers and life science investment fund analysts use this historical statistical data to make predictive estimates of a development program. These approaches have many limitations. On the one hand, they do not consider the multitude of risk factors, and on the other hand, they tend to generalize, whereas empirical observations show that many factors such as the therapeutic area, the research site, the size of the company (Pharma vs. biotech), the experience of the company in clinical development in the therapeutic area concerned, etc. impacted the probability of transition and regulatory success.

### Reasons for Failure in Clinical Development

Reasons for failure in clinical trials are usually automatically classified as the result of the inability to demonstrate efficacy or to ensure the safety of the drug candidate. The issues of efficacy and safety are most often evoked to quickly justify the causes of failure. Efficacy here is understood as the demonstration of the pharmacological action (mechanism of action) expected from the molecule under study. As for safety, it is understood as undesirable pharmacological reactions. Therefore, failures in clinical development are often categorized in a binary way as a reason for efficacy or safety.

While it is not inaccurate to categorize the causes of clinical trial failures in this way, but it can be reductive. Indeed, when it comes to efficacy, it's often a phenotypic conclusion that is due to many other underlying causes unrelated to the mechanism of action. Many other factors related to operational, strategic, or even economic decisions during the clinical development process can result in development failure (Fogel, 2018; Harrison, 2016). Factors such as participant recruitment and enrollment rates, high dropout rates, research site selection, protocol adherence, lack of funding, complexity of research protocols etc. are potentially significant contributors to clinical study failure.

### Patient Recruitment

It is well documented that a low participant retention rate (drop-out) or sub-optimal participant recruitment rate can significantly affect the statistical power of the clinical trial and, in turn, handicap any possibility of demonstrating efficacy and thus result in the failure of the clinical trial. Specifically, studies have estimated that when a trial experiences multiple dropouts, the trial may lose statistical power and result in the inability to demonstrate efficacy. (Fogel, 2018; Hwang et al., 2016; Schroen et al., 2010). For example, a complex Phase II clinical development study protocol taking place at a clinical research site where the principal investigator has a high solicitation rate with few available resources and the site does not have a sufficient pool of eligible patients to ensure a high recruitment rate to maintain necessary statistical power will result in a significantly increased risk of study failure. This can have significant financial consequences for the organizations sponsoring the trial.

Currently, it is estimated that between $600,000 and $8 million in revenue is lost for every day a clinical trial is delayed (CenterWatch, n.d.). To emphasize the complexity of risk in clinical development, it has even been shown that the enthusiasm of the principal investigator at the study site was the most important factor associated with positive enrollment at 60 study sites in a trial evaluating the treatment of local postoperative pain (Fogel, 2018; Fouad et al., 2013). In addition, data show that slow recruitment may stem from inadequate staffing and poor prioritization of clinical trials over daily operations (Thoma et al., 2010)

### Adherence to Treatment

Patient nonadherence is one of the most complex problems facing clinical development. A small degree of nonadherence can have a significant effect on the sample size needed to detect a difference between groups. For example, the literature has shown that a 20% to 30% decrease in adherence may require an increase in sample size of more than 50% to maintain equivalent power. (Serebruany et al., 2005; Smith, 2012). A non-adherence rate of 40% would require tripling the sample size (Serebruany et al., 2005). The direct consequence of such a situation is increased time and cost for the clinical development project of the product under study.

### The Complexity of Research Protocols and Choice of Site Research

The choice of inclusion and exclusion criteria can affect the duration and cost of a clinical trial (Babbs, 2014). The complexity of research protocols affects the likelihood that the clinical trial will achieve the desired levels of recruitment and retention of participants to be able to meet the statistical objectives (Power B) of the efficacy endpoints. For example, in one study, it was noted that out of 3,400 clinical trials, more than 40% that had protocol amendments prior to the first participant's first visit delayed the trials by 4 months (Getz et al., 2011). While some protocol amendments are unavoidable, however, the risk of protocol amendments before the trial begins can be reduced with better planning and anticipation of the consequences of clinical trial design choices. In addition, protocols that are both scientifically and operationally complex are associated with low recruitment and retention rates of participants in clinical trials (Lamberti et al., 2012).

A study reported that 16% of protocol amendments were due to a change in inclusion and/or exclusion criteria, which could result in differences in patient populations before and after the amendment. Also, the same authors reported correlations between the experience gained from the positive results of previous clinical trials and the results of subsequent clinical trials obtained. In this case, the data show that a site with 6 to 10 clinical trials has a greater likelihood of meeting enrollment criteria within the required time frame compared to a site with fewer prior trials. Even considering the most widely known problem in clinical trials, low clinical trial enrollment, it has been shown that research sites with a history of successful performance are historically more likely to meet enrollment goals (Getz et al., 2011). Developing an approach that focuses solely on recruitment performance while overlooking, for example, that the enthusiasm of research staff and particularly the principal investigator may be an important risk factor will not be a winning strategy.

### Regulatory Agencies and Drug Evaluation

In addition to the ever-increasing development costs combined with high attrition rates in clinical trials, the pharmaceutical industry environment is also characterized by increasing requirements by regulatory agencies for the marketing of drugs (Canada: Health Canada; USA: US Food and Drug Administration (FDA); Europe: European Medicines Agency (EMEA) etc.). This is complemented by growing challenges in obtaining reimbursement of products by public reimbursement agencies, which is an asset for the sale of drugs in Canada, Europe, and certain Asian countries, for example, where the requirements for demonstrating therapeutic and economic value are increasingly high. It is important to remember that the productivity of the pharmaceutical pipeline is defined as the ratio between the value (therapeutic and commercial) created by a new drug and the investments required to generate this drug.

Indeed, some geographic regions require more investment to bring a drug to market, both in terms of time and money. Particularly in Canada, the drug evaluation and commercialization processes are considered arduous: it is “fragmented and sequential”. (Hoskyn, n.d.). Because the process of approving a drug for commercialization is separate from the reimbursement process, the jurisdictions that provide market authorization evaluate the drug from a different perspective than the reimbursement review agency. In general, to receive a Notice of Compliance and be able to market a drug, the drug is evaluated individually based on the clinical trials conducted (efficacy, safety and quality). However, during the reimbursement assessment, the drug is compared to all existing options in that jurisdiction to determine its economic and societal value, in addition to its therapeutic value. The drug comparators retained in the comparative analysis by the agencies may vary according to clinical practice, as may the final price recommendation by the health technology assessment agency, which may therefore be disappointing for the manufacturer as the outcome of the evaluation can be unexpected. Therefore, if the public market potential for that particular molecule is important, the manufacturer must decide whether it is willing to negotiate the public price downward to meet the agency's expectations or retain only the private market with a higher price. For some drugs, a private market only does not pose a threat to commercial success where the returns on investment exceed the cost of development. However, this does not apply to all drugs. For example, for a drug that is indicated for a disease affecting patients 60 years of age and older, a private market only is a very large commercial failure since patients 65 years of age and older are generally on the public plan, indicating that the target population of the drug will not have access to the drug and therefore the sales initially anticipated by the pharmaceutical company will not be achieved. In Canada, for a new patented drug, the review process is segmented into 5 general steps, starting with regulatory approval for marketing by Health Canada based on the efficacy, safety and quality of the drug individually based on clinical trials conducted. The patented drug then goes through a price regulation review under the Patented Medicine Prices Review Board (PMPRB) to ensure that patented medicines are not being sold in Canada at excessive prices. The evaluation of the drug is followed by an analysis of the therapeutic value and value to the health care system/societal value by the INESSS (Institut National D′excellence en Santé et Services Sociaux) in Quebec or CADTH (Canadian Agency for Drugs and Technologies in Health) in the rest of Canada. These evaluation agencies issue three possible recommendations: reimbursement without conditions, reimbursement with conditions or no reimbursement. Following a positive recommendation from the health technology assessment agencies, if there is interest from the provinces, a negotiation of price and clinical conditions will take place with the pCPA (Pan-Canadian Pharmaceutical Alliance). Canadian provinces that wish to participate in the negotiation will participate in the discussions. This is with the objective of having equity of access to the drug and homogenization of the national price, among others. A successful negotiation will result in the introduction of the drug to the provincial public drug formularies. However, if the negotiation is not concluded, the drug will not be available for reimbursement in the public plan, only potentially in the private market. The final step following the discussion of the clinical conditions and price of the pCPA process is the signing of confidential agreements with the participating provinces. These provinces will continue with the implementation of public reimbursement at their own pace. This entire approval and reimbursement process can easily take more than three years. Canadian provinces are all independent in terms of budgets and the particularities of their own health care systems. To add an additional layer of complexity, when negotiations are completed at the pCPA, these clinical conditions can evolve to be even more restrictive when it comes to the confidential individual agreement stage with the provinces.

Canada is by no means the only country with a public health system that evaluates public reimbursement, but it is the one with the longest processes and the most heterogeneous decisions. According to a publication by Innovative Medicines Canada that compares Canada with other international jurisdictions, Canada's rate of new molecule introduction is of 65% compared to 96% for 20 Organization for Economic Co-operation and Development (OECD) countries that also have a public reimbursement system (Hoskyn, n.d.). Given that Canada is composed of 10 provinces and 3 territories with different budgets and health care systems, the national reimbursement rate for the same product falls to 53%, highlighting a large variability in reimbursement decisions at the provincial level. Even when the drug has been approved elsewhere in the world, in terms of the length of the reimbursement process, Canada ranks second to last out of 20 OECD countries, with 559 days of review for Canada compared to 138 days for Italy, 13 days for Germany or 32 days for England (Hoskyn, n.d.)

The more innovative and differentiated the drug, the more challenging it is to meet the CADTH, INESSS, PMPRB, and pCPA requirements. It is not enough to successfully complete clinical trials and obtain a marketing authorization. In anticipation of CADTH and INESSS recommendations, these strategic study design decisions need to be thought out in advance. For successful commercialization in Canada, there is a strong need to think about clinical trial design as early as Phase II.

These pharmaco-economic evaluations are governed by submission requirements and evaluation criteria specific to each jurisdiction. In the face of ongoing drug innovation in terms of administration (e.g., subcutaneous, intravenous, oral, etc.), patient type/subtype (e.g., gene-specific), rate of disease progression, companion tests, etc., product submission guidelines and requirements are also evolving. Since 2020, CADTH, the agency that assesses the value of drugs in Canada (excluding Quebec), has issued more than three notices of procedural changes, submission requirements, or notices of fee increases of the review process. This is accompanied by a failure to finalize and implement reforms to the PMPRB's procedures (the body that evaluates the non-excessive prices of patented drugs in Canada) since 2019, which greatly influence product launch decisions in Canada. This indicates that in countries with a health system where drugs are publicly reimbursed, obtaining a Notice of Compliance for a drug is far from sufficient to define commercial success as there are many external factors that can tip the scale.

### Financial Factors

In addition to the efficacy of the drug, administrative factors can have a great impact, such as a poor financial assessment of the capital requirements necessary to ensure the proper conduct of a clinical development project. The financial factor is of crucial importance and its assessment throughout the development process must be done by considering all the contributing factors. Indeed, it has been shown that 22% of failed Phase III studies were unsuccessful due to lack of funding (Hwang et al., 2016). The costs required to complete the entire development process from discovery to market for a drug vary, as do the estimates of those costs. Indeed, considering only Phase III trials, the pharmaceutical Research and Manufacturers of America (PhMRA) estimated the cost per patient to be $42,000 in 2013 with $10 billion spent on 1,680 Phase III clinical trials involving more than 600,000 patients.

### Increase in Public Spending on Medicines.

Indeed, the growing importance of drugs in terms of public expenditure in health care systems around the world reinforces the increasing interest of public authorities in controlling expenditure. Indeed, the cost of drugs is now the single most important input into the production of health care in almost all health care systems throughout the most industrialized countries. The growth in public spending on drugs in the OECD countries is a clear illustration of this importance. In Canada, in 2017, public spending on drugs accounted for 16.4% of total health care spending. The same trend can be observed in the most industrialized countries (e.g., the United States, Austria, Switzerland, Greece, etc.). With the ever-increasing price of drugs, controlling drug expenditures is becoming the most important issue for health system managers and political leaders in industrialized countries. In this case, the willingness of the Canadian government, through its semi-judicial body, the PMPRB (Patented Medicine Prices Review Board), to implement stricter control measures is a strong indication of the changing environmental dynamics. The PMPRB's objective is to establish a ceiling price for patented medicines in Canada. This PMPRB pre-set price will be further reduced by the Pan-Canadian Pharmaceutical Alliance (pCPA) in provincial negotiations. The growing interest of government agencies in drug expenditure control issues is contributing to the complexity of the business environment for the pharmaceutical industry in markets such as Canada, which is sensitive to reimbursement issues as sales are heavily dependent on them.

Not surprisingly, the competitive environment of the pharmaceutical industry is constantly changing. Fundamentally, the business model of the innovative pharmaceutical industry is based on innovation, which is the result of lengthy research and development (R&D) processes that lead to the development of new drugs. This business model has evolved into one that favors mergers and acquisitions rather than huge investments in R&D. But faced with the financial challenges resulting from low pipeline productivity and patent cliffs, it is still difficult for companies to develop new drugs. As a result, innovation remains the keystone of the industry's business model.

The consequences of this situation can be listed on an economic, social and even environmental level. On the economic level, the colossal investments in R&D versus a very high failure rate of molecules in development, sometimes at an advanced stage, are driving up the prices of drugs that reach the market. In recent years, the rising prices of patented drugs have been the subject of ongoing debate. It must be said that the business model of innovative pharmaceutical companies would not be viable if the huge losses resulting from the failure of molecules in development were not compensated by the benefits of the marketing of approved molecules. From a social point of view, three consequences can be envisaged directly from this situation, firstly, patients in need are deprived of quality medicines because of the colossal costs that are unbearable for public payers. Second, the “unnecessary” exposure of participants in failed clinical studies cannot be ignored and is fundamentally ethically problematic (Williams et al., 2015), and finally, the possible loss of jobs for industry professionals that may result. Environmentally, a clinical study requires a lot of resource and material expenditures.

With a pipeline productivity crisis supported by an increasing cost of developing new drugs combined with a high failure rate, the pharmaceutical industry has been told that with such statistics, the business model on which this industry is based could not be sustainable in the long term. Indeed, risk is inherent to the pharmaceutical industry by its primary mission to innovate. But pharmaceutical innovation has specific characteristics as mentioned above that make it a very risky business. One would have thought that with such particularities the pharmaceutical industry would be equipped with powerful strategic pharmaceutical portfolio management tools to overcome these problems that have plagued the industry for decades. The pharmaceutical industry has been very slow in developing and implementing such tools (Kwak & Dixon, 2008). It is therefore vital for this industry to continuously develop new strategies to optimize risk. An analysis by the consulting firm Mckinsey & Co. points out the important need for the pharmaceutical industry to develop approaches inspired by other business sectors such as banking and finance. Moreover, the analysis predicts that future years will be even more difficult for this industry without the implementation of new risk control approaches (Dhankhar et al., 2018). The issue of R&D efficiency in the pharmaceutical industry characterized by its very meager output has already been widely discussed. Indeed, some authors define R&D efficiency as the successful approval and launch of new drugs relative to the proportion of monetary investment required for R&D (Schuhmacher et al., 2016). This efficiency has declined dramatically for decades. When one considers that R&D investment has nearly exploded in recent years, it is therefore legitimate to ask what effective ways are to increase pipeline efficiency in this industry.

Conceptually, risk is a complex notion due to the variation in adaptability depending on the field of activity. Thus, whether in health, finance, technology, environment, cyber security, defense, etc., risk is not understood in the same way. In medicine, we talk about medical risk; in finance, we talk about financial risk; in the environment, we talk about the environmental risk of an oil pipeline project, for example, in manufacturing, we focus on eliminating the risk of defects in the production line, etc.

According to Cornelius Keating, risk is the undesirable subset of a set of uncertain outcomes. Indeed, the research revealed that there was no consistent definition of risk and that it depended on the sector. However, the notion of uncertainty seems to be inherent to the notion of risk. In fact, the International Organization for Standardization standard ISO/ISO 31000 (2009)/ISO Guide 73:2002 defines risk as “the effect of uncertainty on objectives”. Uncertainty is intrinsically linked to the notion of probabilities. Basically, risk management is the systematic process of identifying, analyzing and responding to project risks. This includes maximizing the probability and consequences of positive events and minimizing the probability and consequences of undesirable events in relation to the project objectives (PMBOK® Guide, n.d.).

In almost all notable industrial spheres, be it aerospace, energy, finance, insurance, etc., risk management is an integral part of management's activity. Nevertheless, as analysts from Mckinsey & Co. point out, risk management is increasingly becoming an issue for all industries and according to them, even more so for the pharmaceutical industry which will face risks especially in the design and execution of clinical trials, drug approval, product quality and global business practices and these risks are growing in frequency and magnitude (Dhankhar et al., 2018). Therefore, it becomes imperative for the pharmaceutical industry to address it more deeply to develop better risk management methods adapted to this industry which has many peculiarities that require a risk management approach and efficient tools adapted to these peculiarities which could be a disadvantage to the adoption of standard approaches.

It has therefore become imperative over the years for the pharmaceutical industry to find new approaches to optimize strategic decision making in pharmaceutical portfolio management. Indeed, like any other organization, the pharmaceutical industry is constantly seeking efficiency and effectiveness in its decision-making processes to optimize the use of its resources. The productivity crisis as defined here represents a major challenge for pharmaceutical portfolio managers.

The nature of risk in the pharmaceutical industry can be understood as the result of scientific, regulatory, and economic uncertainty (Dickson & Gagnon, 2004). Further analysis allows us to understand that many factors can explain the high failures in drug development.

### Traditional Risk Control and Portfolio Management Approaches to Drug Development

Pharmaceutical portfolio managers, clinical development project managers, life science investment fund managers, contract research and clinical development organizations (CROs) use statistical approaches to make risk estimates. Particularly, existing historical clinical trial data allows for statistical calculations of phase transition odds. This statistical data combined with the opinions of industry experts who, through the strength of their knowledge accumulated over the years, can make recommendations on the conduct of individual clinical development projects.

The pharmaceutical industry environment is characterized by its dynamic, rapidly changing regulatory and commercial environment, long research, and development (R&D) lead times and the complexity of the development process. Statistical data is therefore limited in its ability to consider these many factors over time. Statistical data provides a historical estimate with no guarantee of generalization over a projected time horizon.

Given these challenges that threaten pharmaceutical innovation, pharmaceutical companies have developed some qualitative and quantitative approaches for risk mitigation strategies in drug development process.

According to Cooper et al., pharmaceutical portfolio management can be defined as a dynamic decision process that involves continuously updating and revising a company's list of active new products and the ongoing R&D projects. This dynamic decision process involves the evaluation, selection, and prioritization of new projects. The ongoing projects can be accelerated, killed, or deprioritized; hence the resources are allocated or reallocated to active projects (Cooper et al., 1997).

The main objective of implementing portfolio management strategies in the pharmaceutical industry is increasing R&D productivity and decreasing risk. R&D productivity can be defined as the ratio of the value (therapeutic and commercial) created by a new drug and the investment required to generate that drug. To achieve these goals, the pharmaceutical industry has developed qualitative and quantitative approaches for risk management in drug development.

Traditionally, pharmaceutical portfolio management has relied on the use of historical estimates of regulatory approval rates and human judgement to support drug development decisions (Betz, 2011; Krishnan & Ulrich, 2001). However, relying on human judgment and historical estimates results in obvious limitations inaccurate results. These estimates are inherently dependent on many factors, including the therapeutic class and the stage of development, as well as the empirical knowledge of Key Opinion Leaders (KOL). Among these qualitative and quantitative traditional decision-making tools, we can cite:

### Financial Models: (e.g., Net Present Value, Internal Rate of Return Etc.)

Certain criteria and tools take from financial mathematics allow pharmaceutical portfolio managers to assess the feasibility of an investment in a development program. These include: The estimation of an unmet medical need. This is generally obtained through a market study. It allows to estimate the size of a market and to anticipate the potential market shares to be acquired. In general, the annual revenues of a product are estimated using the current sales of drugs used to treat similar indications.
a. Commercial value.
b. Net Present Value (NPV). The value of future cash flows after discounting to today's money. NPV=x/(1+k) n;
c. The net cash flow (x) is discounted annually at the discount rate (k) and is paid in n years.
d. Probability of successful launch (P);
e. Development costs (C);
f. Risk-adjusted cost rC=C×P;
g. Productivity index PI=NPV/C;
h. Risk-adjusted productivity index=rNPV/rC;
i. Risk-adjusted net present value. It can be roughly expressed as: rNPV=NPV×P.

Net Present value of a program when revenues, risks, costs and time are all taken into account; allows comparison over a heterogeneous portfolio of candidates. It is more accurate than NPV in the context of the pharmaceutical industry where the risk components are particularly dependent on many factors. The NPV is therefore adjusted (multiplied) by the probability of success of the launch to take this into account.

Specifically, it is the difference between the NPV of the projected gain adjusted by the current probability of success P minus the sum of the NPV of costs adjusted by the relative risk after i periods.

$$rNPV = NPV ( G * Po ) - {\sum }_{k = 0}^{n} NPV ( {C}_{k} \frac{{P}_{o}}{{P}_{k}} )$$

Probabilistic financial models: (e.g., Monte Carlo Simulation and decision trees).

Strategic approaches: Selection of projects are largely driven by the business strategies.

Scoring models: Projects are rated and scored on a variety of qualitative criteria.

Mapping approaches: (e.g., Boston Consulting Group portfolio models).

However, despite years of implementation of these approaches in drug discovery and development, the problem of high attrition levels persists. Some authors identified the factors that are mainly considered to construct an optimal pipeline portfolio, including the cost of development, the likelihood of surviving, the expected profitability, the competitive landscape, the market size, and the novelty of the drug (Ding & Eliashberg, 2002; Jekunen, 2014).

Indeed, while it is well known that the drug development process is time consuming, expensive, complex, and highly risky, the nature of risk in the pharmaceutical industry is multidimensional. Despite pharmacological efficacy and safety being very well-known failure factors of drug development, the literature abounds with a multitude of other important failure risk factors of a strategic, operational, and commercial nature. The decisions associated with these risk factors are major contributors to failure in late stages and are difficult for humans to control (Sorintellis, 2022). An important limitation of these approaches is the accuracy of the value of the estimator of the probability of the successful launch of a development program regardless of its stage of progress by considering all potential risk factors and providing the possibility of continuous reassessment given the temporal component that is strongly associated with clinical development.

With the advent of Artificial Intelligence, a few approaches in drug development were developed such as those facilitating the discovery and design of new molecules (Spencer et al., 2015; Stokes et al., 2020), improving the structural profile of molecules (Dahl et al., 2014), repositioning old molecules (Ashburn & Thor, 2004; Klaeger et al., 2017) and improving pharmacovigilance (Kompa et al., 2022), among others.

### Artificial Intelligence (AI) and Predictions of Clinical Transition Phases

The advent of AI and more particularly of Machine Learning techniques allows to overcome the limitations associated with statistical estimation methods. Machine learning, which can be defined as the ability to learn without explicitly programming it, requires the combination of three essential factors: Data, computational power, and algorithms/models. The availability of huge data sources accumulated over the years on clinical research projects is an indispensable asset for the development of machine learning algorithms.

A few publications demonstrate the possibility of making predictions of failure but applied to a subspace of potential risks. It is uncertain whether the methods proposed in this literature are applicable to the business scenario under consideration, particularly for the evolution of clinical trials in Canada, where additional approvals are required.

In the literature, the definition of success of a clinical trial varies depending on the articles and does not take into account all the operative constraints such as trust, decision-making, etc. The current definition does not take into consideration other dimensions, such as simplified approaches for the sake of competition/publication with a single target variable (mono-objective) while there is added value in modeling all the response variables.

In this case, by definition, commercial success in a health care system such as the US would be associated with marketing approval (MA), i.e., obtaining regulatory approval to market from the US Food and Drug Administration (US FDA). In a Canadian context, where the health care system has a pronounced public reimbursement component (INESSS, CADTH), commercial success is defined as not only obtaining marketing approval (Health Canada), but also having the drug added to the public insurance reimbursement lists. Therefore, in such a case, it would create even more value for the industry if, in addition to predicting the transition from clinical phase to regulatory approval, we could also predict the success rate of listing on reimbursement lists. Currently, publications that demonstrate the ability to make predictions of failure focus on a specific scenario with no certainty of applicability in a Canadian or European context. Moreover, the publications are circumscribed in an American geography (FDA), here we consider extending the prediction of regulatory approval on other legal geographies as in the European Union (EMA: European Medicines Agency), Canada (Health Canada). In addition, there are many diverse causes of failure, which are not necessarily identified and annotated to clinical trials in public data sources. Prediction of failure may therefore need to incorporate subtle interactions between multiple variables, which is not realistic for a human. A functional tool must be able to be used for multiple types of clinical trials, and acquiring knowledge specific to each type e.g., to identify at-risk patterns, is not viable at large scale. A reproducible machine learning strategy is therefore needed for inclusion in multiple clinical trial types.

However, these methods only take into consideration clinical research data, and therefore, focus specifically on clinical risk. On the other hand, if they allow to give a score/probability of success for a clinical study, they do not provide in a more practical way, a capacity of dynamic follow-up of the clinical study in the duration. This last fact is fundamental in the daily practice in the pharmaceutical industry on the one hand because a clinical study takes place prospectively over a long period of time and the follow-up of the events impacting the success probability score is crucial for a better risk control.

Furthermore, no current method allows for the prediction of the approval of a drug for reimbursement in publicly funded health systems. All current prediction methods are limited to the clinical transition and regulatory (FDA) approval phases.

### SUMMARY OF THE INVENTION

The aforesaid and other objectives of the present invention are realized by generally a system, method, and platform applicable to the specific areas of strategic pharmaceutical portfolio management, regulatory affairs, strategic management of business processes in the clinical drug development phases, pharmaco-economics, investment strategy optimization for life sciences venture capital funds, risk management, due diligence for pharmaceutical mergers and acquisitions, and the stock market. This platform operates in an environment of heterogeneous data from multiple open and private sources. The gathering, organization, enriching by manual labeling by domain experts, processing, and analysis of these millions of data points by artificial intelligence algorithms (Machine Learning), allows to generate predictive and prescriptive actionable insights for a better a priori and continuous control of risk factors to efficiently optimize decisions in the strategic management of the pharmaceutical portfolio as well as the conduct of clinical trials and in all decision-making recommendations in the other areas mentioned above.

In an aspect of the invention, a system for predicting level of success of a clinical trial of a pharmacological product is provided. The system comprises a data source comprising data relating to the clinical trial, and a server. The server comprises a data acquisition module in data communication with a plurality of external data sources comprising data relating to clinical trials, regulatory approvals, economic and reimbursement information, pharmacological information, and commercial and corporate information. The server comprises a data processing engine configured to transform and normalize data from the data acquisition module using a natural language processor. The server comprises a machine learning engine comprising a model trained with the data processed by the data processing engine, the trained machine learning engine being configured to execute an algorithm to analyse the data of the data source relating to the clinical trial and to calculate a prediction of success of the clinical trial based on the said analyzed data.

The model being trained by the machine learning engine may further be a multi-tiers model. The multi-tiers model may further comprise a first-tier model configured to calculate a plurality of intermediate predictions of success, and/or a plurality of first tier models, each model being configured to calculate an intermediate prediction of success. The plurality of first tier models may further comprise at least one of a model to calculate prediction of target recruitment of the clinical trial, a model to calculate a prediction of protocol deviation of the clinical trial, and a model to calculate other factors relating to the clinical trials. The intermediate prediction of success of each of the first-tier models may further be inputted in second-tier model to calculate the prediction of the success of the clinical trial.

The system may further comprise a module to interpret and explain the calculated prediction of success of the clinical trial. The module to interpret and explain the calculated prediction of success of the clinical trial may further comprise generating logical rules used to calculate the prediction, and any of a set of contribution attributes of the clinical trials, studies used to compare to the clinical trial, contrasting explanations and scenarios impacting level of predicted success of the clinical trial.

The system may further comprise an application module configured to execute the machine learning engine with data relating to the clinical trial, wherein the acquired data source may be classified in a plurality of repositories. These repositories may further comprise any one of the following type of data: clinical data, regulatory data, economic data, molecule data and MPP.

In another aspect of the invention, a computer-implemented method for predicting level of success of a clinical trial of a pharmacological product is provided. The method comprises acquiring data from a plurality of external data sources comprising data relating to clinical trials, regulatory approvals, economic and reimbursement information, pharmacological information, and commercial and corporate information. The method further comprises processing, transforming and normalizing the acquired data using a natural language processor. The method further comprises executing a machine learning model trained with the processed, transformed and normalized data to analyse data relating to the clinical trial and to calculate a prediction of success of the clinical trial based on the said analyzed data.

The trained model of the method may be a multi-tiers model comprising a plurality of first-tier models and a second-tier model, the method further comprising each of the first-tier model calculating an intermediate prediction of success of a specific aspect of the clinical study.

The method may further allow the plurality of first tier models to calculate one of a prediction of target recruitment of the clinical trial, a prediction of protocol deviation of the clinical trial or other factors relating to the clinical trials. The second-tier model may use each of the intermediate predications calculated by the first-tier models to calculate the prediction of success of the clinical trial.

The method may further comprise monitoring in real-time progress characteristics of the clinical study using such characteristics to calculate the prediction of success of the clinical trial. The characteristics may further comprise anticipation of recruitment needs and identification of impacting events.

The method may further comprise the execution of the machine learning model further calculating any one of the followings: clinical risk of the clinical trial, regulatory risk of the clinical trial, commercial risk of the clinical trial and pharmacological risk of the clinical trial. The execution of the machine learning model may further generate prescriptive data for optimizing study conduct.

The method may further comprise developing a plurality of machine learning model for the clinical trial, training the developed models with acquired data and selecting one or more of the developed models based on performance metrics.

In yet another aspect of the invention, a computer-readable medium storing instructions for executing the above method is provided.

### An Example of Application

Enhanced criteria for a priori estimation of the evolution of success (regulatory approval and/or public reimbursement) of a molecule under study while it is still in the early stages of clinical development.

Our invention concerning a platform, system or method generally aims at answering the major problem associated with the efficiency of the pharmaceutical pipeline productivity. The platform, system or method generally aims to optimize strategic decision-making in the management of the pharmaceutical portfolio. In this case, the platform, system or method may be specifically aimed at the collection, integration and dynamic and continuous analysis of an exhaustive risk factors impacting the estimation of clinical development and commercial success of a drug study. On the other hand, the platform, the system or the method allows to estimate and classify the decisional risk factors allowing an optimal management of the pharmaceutical pipeline for molecules in clinical development phase (phase I, II and III). The knowledge thus obtained allows to evaluate and optimize very early the commercial success of a molecule under study.

The drug development process can be segmented technically and temporally as a sequence of two major stages, the discovery phase and the clinical development phase. Given that, despite numerous technological advances developed to improve the chances of success in the discovery phase, and the current persistence of still high attrition rates in the clinical development phase; Given the understanding that, apart from efficacy and safety issues (pharmacological factors), the strategic, operational and even commercial decisions are also important causes of failure in the late phases (phase II and III) of clinical development; Given the significant unsustainable costs associated with late phase failures as compared to early discovery phases; Given that current methods based on artificial intelligence aim at optimizing the structure-activity-molecule relationship in the early discovery phases in order to remedy the causes of efficacy and safety failures (pharmacological factors); The need for a system that takes into account the equally important risk factors, i.e. strategic, operational, commercial decisions in late phase clinical development brings a useful, necessary and relevant inventive contribution.

The additional inventive contribution of basing the platform, system or method on artificial intelligence (Machine Learning, Deep Learning, NLP, etc.) allows for the dynamic collection, integration and real-time analysis of vast sources of varied data, whether structured or unstructured, and thus goes beyond the immutable and non-evolving statistical estimates of current methods. The usefulness of dynamic and continuous analysis is based on the fact that the pharmaceutical industry environment is constantly changing and that it takes many years to develop a drug. Therefore, a method that takes into account temporal factors and continuously integrates data allows for a tenfold increase in analytical capacity and optimized decisions, as opposed to statistical data that is fixed over time. Moreover, the platform, system or method is intended to provide both predictive and prescriptive data. The prescriptive data allows to take actions to improve the chances of success but also to continuously re-evaluate the decisions already taken.

The platform, system or method will make it possible for the users to receive predictions based on features available at each clinical phase and moreover, our approach will be equipped with counterfactual analysis that generates recommendations on how to parameterize the clinical study to have an acceptable prediction of success. All these models and the counterfactual explanations should be served to users via a user interface. This way the user will have access to a set of diverse trained models for different phases of clinical trials, along with explanations on models' decisions. Therefore, the user can assess the impacting factors, tune them as much as possible, and assess the success predictions. Finally, methods and systems will generate these insights and deploying them in an understandable manner to guide strategic decision-making in pharmaceutical portfolio management.

The system is built around a major risk modules structure, including clinical development risks which include operational risks, regulatory risk, economic or financial risks and pharmacological risks (inherent to the molecule under study). The output allows the generation of predictive and prescriptive insights from drug clinical development including clinical study startup to market access and commercial success based on a priori estimate of the multidimensional risks associated with the development of a drug candidate. For different stakeholders such as clinical research project managers, pharmaceutical pipeline managers, clinical research organizations (CRO), life sciences venture capital analysts' dynamic prescriptive information on ongoing clinical development projects will help strategic, operational or commercial or business decisions that may result in failure if not adequately controlled.

The historical data which could be structured or unstructured used come from existing public and private databases resulting in millions of data points from an aggregate of multivariate data enriched by manual labeling processing by domain experts. Their gathering and integration allowing an in-depth drug development risk network understanding and mapping,

Thus, the process defined here considers as cardinal foundations of major pillars capturing the process in its entirety, including pharmacological risk, clinical development risk which include operational risk, regulatory risk, economic or financials risk and strategic risks.

### The Overall Clinical Risk

It is defined in this invention as the overall risk associated with conducting clinical development processes. The clinical risk is estimated by taking into account all micro elements of clinical risk contributing to it over time and depending on their intensity. These micro elements of risk that contribute to the estimation of the overall clinical risk interconnect in a dynamic and evolving interdependent sub-network. More precisely, it allows to assess the clinical risk regardless of the phase of development, the therapeutic field, the research site, the study protocol, etc., and by integrating all its factors together, rather than treating them individually as it is traditionally practiced. Moreover, the process based on machine learning algorithms will allow to efficiently predict this global clinical risk factor with a good understanding of the prescriptive information to take corrective actions to improve the predictive score hence the dynamic nature of the process. This includes clinical trial decision elements that are administrative and operational in nature, such as Principal investigator selection, geographic location of the trial, site selection, contract agreement etc.

### The Overall Pharmacological Risk

The overall pharmacological risk is defined here as the risk inherent to the molecule. This includes, for example, the known pharmacological characteristics related to the molecule under study. Knowledge about the molecule under study is of major importance in drug development. Paul Ehrlich in his famous quote Corpora non agunt nisi fixata (drugs will not act unless they are bound) thus underlined the importance of the molecular activity in the understanding of the pharmacological activity. In drug development, the quantitative structure-activity relationship (QSAR) is the process by which a chemical structure is correlated with a well-determined effect such as biological activity or chemical reactivity. It is therefore even more useful as it allows to explain and anticipate pharmacological properties such as the activity of a compound on a target receptor, including the efficacy and even the safety of potential drugs. Taking this risk factor into consideration is important since we know, for example, that a chemical molecule does not have the same probability of success as a biological molecule.

### Global Regulatory Risk

Global regulatory risk is defined here as the risk of success (marketing approval) at the regulatory level based on clinical phase data, such as approvals by Health Canada (Canada), Food and Drug Administration FDA (USA), European Medicines Agency EMA (Europe) etc. Depending on the health care system in place, whether patient access to drugs is through public and/or private plans, regulatory approval is the initial step in determining commercial success. The pace of introduction of innovative drugs is slower than a few decades ago. Drugs entering the market need to differentiate themselves significantly from the competition, for example through more complex mechanisms of action. Clinical trial requirements must adapt to evolving mechanisms of action and may create unconventional clinical trials or measures. In the face of change, the requirements for submitting drugs for approval may also evolve. The method and system can help uncover the risk factors for approval failure by compiling historical results, considering changes in requirements in real time and determining a regulatory success rate.

### Global Economic Risk

This module is applicable to geographic jurisdictions where reimbursement authorities are present and play a key role in the marketing of the drug (such as Canada, Europe, Asia, Australia, etc.). Typically, in these countries, drugs cannot be distributed under the public drug plan without an evaluation of the product and negotiation of clinical conditions and/or price. In this context, for countries with a public health system and depending on the characteristics of the target population of the products, commercial success depends on obtaining reimbursement with a certain price and certain clinical conditions, for example.

The barrier to entry that is the reimbursement process in Canada may deter manufacturers from pursuing with commercialization of a drug, therefore limiting medication access in Canada vs the rest of the world.

Stakeholders in the Canadian health care system are aware of the great misalignment in the drug review process with the various sequential stakeholders. There are efforts to align processes and reduce duplication of work. With the election comes new promises, such as the creation of a national drug plan rather than one fragmented into provinces. This suggests further changes to regulation, access to drugs and pricing.

The system, by its dynamic nature and capturing changes in real time, will help monitor and alert to changes in study design strategy or clinical practice by informing on new molecules under review by regulatory agencies (Health Canada, FDA, EMA, etc.), changes in policies and requirements of reimbursement regulatory agencies, changes in drug prices (incl. international drug prices), reasons for reimbursement/non-reimbursement of drugs, drug evaluation timelines, submission criteria, among others. This information contributes to the strategic decisions that are crucial to determine the commercial success of a drug and increase the pharmaceutical productivity rate.

Referring now to FIG. 1, an in-depth drug development risk network understanding and mapping is illustrated. The perspectives according to the business needs of three important stakeholders are illustrated. The stake holders are contract research organization (CRO), pharmaceutical companies and biotech, and life sciences venture capital.

From the perspective of a CRO, the success of the clinical trial of any phase comes down to the efficient execution of a clinical trial from the start of the trial until the “Last Patient Last Visit” (LPLV) milestone.

From the perspective of an innovative pharmaceutical company, fundamentally, as a first step the success of the clinical trial of any phase is obtaining positive clinical or statistical results in addition to the efficient execution of a clinical trial i.e., the start of the trial until LPLV. A development program is successful when the LPLV status and positive results are achieved in combination with obtaining marketing authorization (Regulatory approval) and showing a positive ROI (commercial success).

From the perspective of a Life Sciences Venture Capital (LSVC), success comes down to obtaining a positive ROI which generally translates into obtaining regulatory approval, hence also showing clinical trial success as defined by the pharmaceutical perspective.

Referring to FIG. 2, based on these different perspectives, the present approach comprises different states of interest transitions for a predictive model. The model comprises three (3) phases (I, II and III), an approval step and a commercial success (CS).

“Concluded” status: State of successful execution of a clinical trial. We will define the intraphase probability as the probability of operational completion of a clinical trial. It applies to all phases f (Ph1); f (Ph2); f (Ph3). For example, the f (Ph2) is the probability that a Phase II study will be performed and completed regardless of the statistical/clinical results. This is the operational success of the clinical trial.

“Concluded+positive outcomes”: State of achievement of successful execution of a clinical trial, both from an operational and statistical/clinical point of view. This is the sine qua non for advancing from one phase to the next. It applies to all Phases I and II, that is, f (Ph1-Ph2); f (Ph2-Ph3). For example, f (Ph1-Ph2) would correspond to the probability of progressing the clinical trial from Phase I to Phase II.

“Regulatory approval” status: Status of regulatory approval. This state is dependent on the successful execution of a clinical trial from both an operational and a statistical/clinical point of view. It applies to all phases f (Ph1-App); f (Ph2-App); f (Ph3-App). For example, f (Ph3-App) will correspond to the probability of obtaining regulatory approval for a given drug in a given indication a priori the launch of the pivotal Phase III trial.

“Commercial Success” status: Commercial success for pharmaceutical products in a health care system such as the United States would be associated mainly with a Marketing Authorization Holder (MAH), i.e., obtaining regulatory approval from US Food and Drug Administration (FDA) to market the product. In a Canadian context and in some European countries, where the health care system has an important public reimbursement component such as INESSS1 in Québec and CADTH2 in the rest of Canada respectively, commercial success is defined not only by obtaining marketing approval, but also by having the drug of interest added to the public insurance reimbursement drug lists (RAMQ3 in Québec). Therefore, in such cases, it would create great value for the industry if, in addition to predicting “Concluded” Status, “Concluded+positive outcomes” and “Regulatory approval” status we could also predict the success rate of listing on reimbursement lists in the Canadian context as explained above, for example. ¹ Institut national d′excellence en santé et en services sociaux (https://www.inesss.qc.ca) ² Canadian agency for drugs and technologies in health (https://www.cadth.ca) ³ https://www.ramq.gouv.qc.ca/fr/a-propos/liste-medicaments

The system is therefore a dynamic, evolving and integrative process that allows the entire clinical drug development process to be encompassed until commercial success.

By leveraging machine learning approaches and an in-depth drug development risk network mapping and millions of data points from an aggregate of multivariate data enriched by manual labeling processing by domain experts, the methods and systems will provide predictive and prescriptive insights from drug clinical development including clinical study startup to market access and commercial success based on a priori estimate of the multidimensional risks associated with the development of a drug candidate. Finally, methods and systems will generate these insights and deploying them in an understandable manner to guide strategic decision-making in pharmaceutical portfolio management

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other objects, features and advantages of the invention will become more readily apparent from the following description, reference being made to the accompanying drawings in which:

FIG. 1 is a diagram of in-depth drug development risk network understanding and mapping.

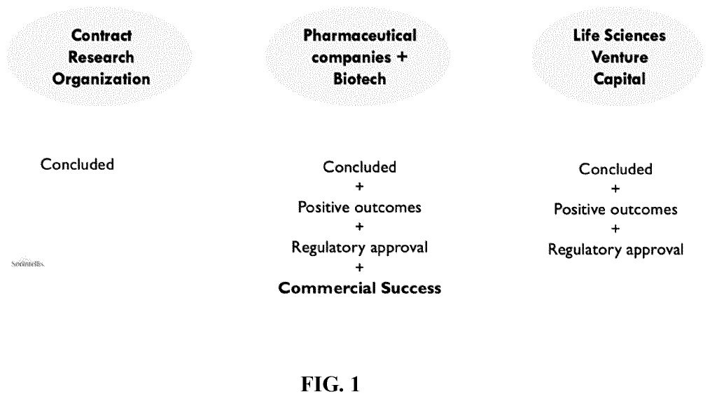

FIG. 2 is a diagram of different states of interest transitions for a predictive model in accordance with the principles of the present invention.

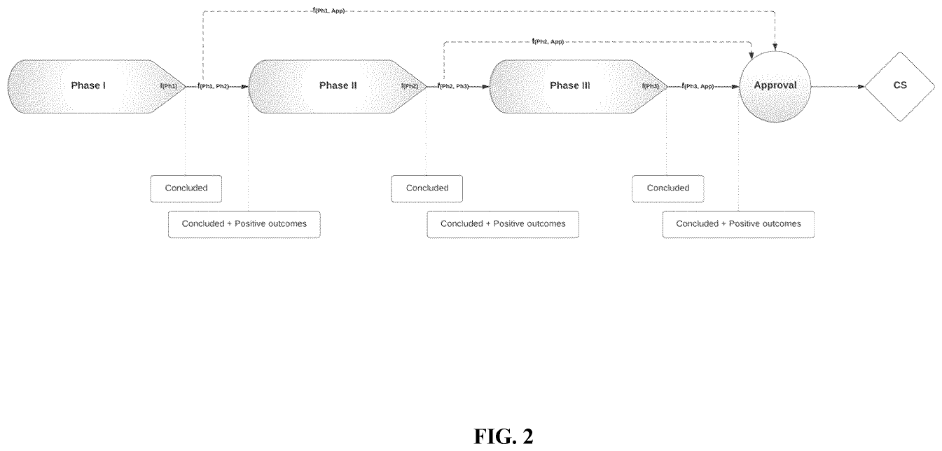

FIG. 3 is an architecture diagram of the high-level architecture of an embodiment of a system and method to generate predictive but also prescriptive information to support strategic decision making a priori and during the clinical drug development phases in accordance with the principles of the present invention. The system comprises three main inter-related components: data sources, a system and the applications.

FIG. 4 is a workflow diagram of the data sources of the system of FIG. 1

FIG. 5 is an architecture diagram of the system based on artificial intelligence algorithms

FIG. 6 is an architecture diagram of the execution and deployment module. The execution and deployment module are implemented as analytical, predictive, and prescriptive applications to support strategic decision making a priori and during the clinical drug development phases.

FIG. 7 is a flowchart of an embodiment of a method for pharmaceutical portfolio strategic management decision support based on artificial intelligence in accordance with the principles of the present invention.

### DETAILED DESCRIPTION OF THE PREFERRED EMBODIMENT

A novel method, and system for pharmaceutical portfolio strategic management decision support based on artificial intelligence will be described hereinafter. Although the invention is described in terms of specific illustrative embodiments, it is to be understood that the embodiments described herein are by way of example only and that the scope of the invention is not intended to be limited thereby.

The invention describes a system, method and platform using artificial intelligence to optimize strategic pharmaceutical portfolio management decision support. The platform is trained on various structured and unstructured data from open and private data sources to generate predictive but also prescriptive information to support strategic decision making during the clinical drug development phases. The preferred embodiments chosen to illustrate the present invention do not limit the scope of the invention.

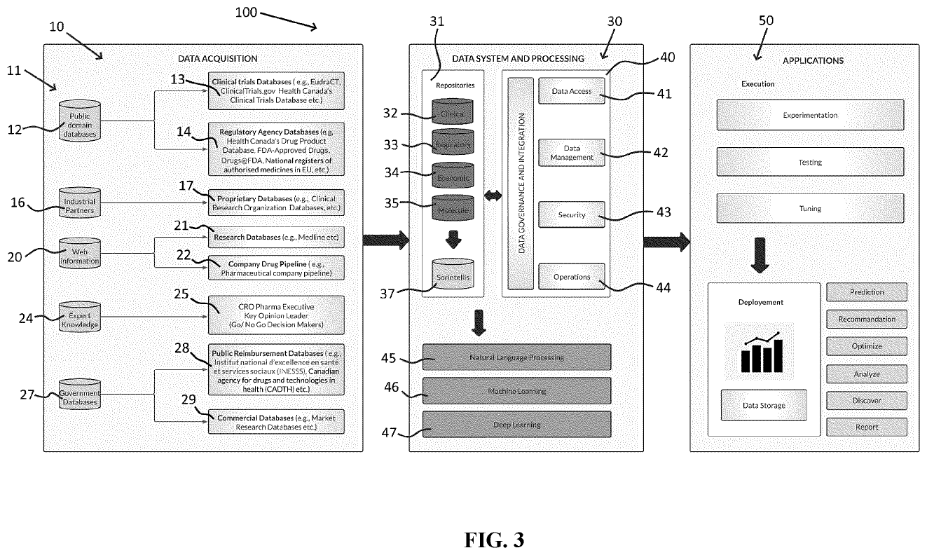

Referring to FIG. 3, an overall high level architectural view of an embodiment of a system for pharmaceutical portfolio strategic management decision support based on artificial intelligence 100 is illustrated. The system is typically implemented as an intelligent platform for generating predictive but also prescriptive information to support strategic decision making during the clinical drug development phases. The architectural structure is divided into three parts: data sources, system, and applications.

The system 100 typically comprises a data acquisition module 10, a data processing module 30 and one or more applicative programs 50. The data acquisition module 10 comprises programs or application to fetch or obtain data records from one or more data sources 11. The programs may use application programming interfaces (API), network protocols or data files to retrieve the data records. The data acquisition module 10 may be configured to connect with public domain databases 12, industrial partners 16, information from the Internet 20, knowledge from experts 24 and/or government or official databases 27.

The data sources 11 may comprise open and non-open databases. In some embodiments, public domain databases 12 may comprise clinical trials databases 13, such as but not limited to www.clinicaltrials.gov, an online database belonging to the United States government containing more than 440,000 clinical trials conducted worldwide. In yet other embodiment, the clinical trial databases may comprise EudraCT (Europe)—EudraCT (European Union Drug Regulating Authorities Clinical Trials Database) contains information on interventional clinical trials on medicines conducted in the European Union (EU), or the European Economic Area (EEA) which started after 1 May 2004. Clinical Trials Database (Canada)—Health Canada's Clinical Trials Database is populated with information about each clinical trial after Health Canada issues the NOL.

The public domain databases 12 may further comprise regulatory agency databases 14, such as but not limited to US FDA, Health Canada's Drug products, EMA, National registers of authorized medicines in EU, etc.

The industrial partners data sources 16 may comprise proprietary databases 17, such as but not limited to clinical research organization databases. The information from the Internet 20 may comprise research databases 21, such as Medline™, company drug pipeline 22, such as pharmaceutical company pipeline. The knowledge from experts' source 24 may comprise CRO Pharma Executive Key Opinion Leader (Go/No go decision makers) 25.

Once all planned clinical trials are completed with positive outcomes for a particular drug-indication, depending on the geographies of interest of the sponsors to market and sell the drug, the full clinical dossier of the drug under evaluation is submitted for evaluation by regulatory agencies to obtain marketing authorization. With slight variations between the level of information contained in these regulatory databases, we can find information about the drug, the indication and the approval date.

The government or official databases 27 may comprise public reimbursement databases 28 such as but not limited to INESS and CADTH databases, and commercial databases 29 such as market research databases.

As mentioned above, upon obtaining marketing authorisation from a regulatory agency, depending on the geographical area where the drug is intended to be sold, additional steps are required to bring the drug to market such as a Health Technology Assessment (HTA) review process. Once a particular drug-indication has received a marketing authorization approval by a regulatory agency, an HTA serves as a multidisciplinary process summarizing information about the medical, social, economic and ethical issues related to the use of a health technology. CADTH Canadian Agency for Drugs & Technologies in Health (CADTH) reimbursement reviews are comprehensive assessments of the clinical effectiveness and cost-effectiveness, as well as patient and clinician perspectives of a drug or drug class. The assessments inform non-binding recommendations that help guide the reimbursement decisions of Canada's federal, provincial, and territorial governments, except for Quebec. The database containing CADTH recommendations is downloadable in CSV and contains 1043 entries. INESSS Institut National d′Excellence en santé et en Services Sociaux (INESSS) is the HTA agency responsible for recommending reimbursement decisions to the Ministry of Health in Québec. Once the Ministry's decision is published, the drug will be listed on the public list of drugs under the general drug insurance plan in Québec in pharmacies or hospitals. A manual extraction of the database is required. The entirety of the database contains 5978 entries as of February 2023. pCPA Following the published positive reimbursement recommendation by CADTH/INESSS, the negotiation process with pan-Canadian Pharmaceutical Alliance (pCPA) is done through four steps with all provincial jurisdictions that are interested to participate in the process: 1—Initiation, 2—Consideration, 3—Negotiation, 4—Completion. These steps come with different sets of deliverables and with different statuses posted on the pCPA website. The 754 entries were extracted manually in an Excel file. European reimbursement landscape: After obtaining marketing authorization from the European Medicines Agency (EMA), the additional therapeutic benefit of the product under evaluation is compared with other new or existing treatments by individual EU member states HTA bodies. A drug can be reimbursed under different rules and prescribed to widely varying degrees within the individual EU countries. The HTA procedure is followed by a price negotiation, which could take place centrally or at the regional level. Indeed, considering adequate planning of the trial design and proper choice of outcomes of pivotal trials are essential since some European jurisdictions do not accept some clinical endpoints for certain therapeutic areas as valid. There are essential considerations to take into account to ensure proper forecasting of market access and sales.

Still referring to FIG. 3, the system 100 is generally configured to organize and process the data acquired from different sources. The system 100 is further configured to process, convert and/or prepare the fetch data from the data sources, also referred as the raw data, to be outputted to artificial intelligence algorithms. In some embodiments, the system 100 is configured to store the prepared and structured data in a data source, such as but not limited to a database, a file or any other storage means. In such an embodiment, the system 100 is configured to execute a program or an application module 50 implementing artificial intelligence algorithms and to feed the prepared and structured data to the said program. The artificial intelligence program 50 is configured to generate predictive but also prescriptive information to support analysis and strategic decision making a priori and during clinical development in the various application areas. The system 100 is mainly based on machine learning.

The data processing module 30 is configured to process the data acquired or obtained by the data acquisition module 10. The processing module 30 is configured to classify the data into a plurality of repositories 31, such as but not limited to clinical data 32, regulatory data 33, economic data 34, molecule data 35, and internal data 37.

The system may comprise an internal data source comprising the internal data 37. The internal data source may be a structured relational database comprising the data from collected and annotated databases and may comprise relationship allowing for the linking of scattered data collected from various databases. As an example, clinical trials are accessible from clinicalTrials.gov and are separately listed in Phase 1, Phase 2, and Phase 3. The clinical trials having progressed from Phase 1 to Phase 3 are not accessible to a layperson or even insiders. The present method allows linking clinical trials for the same drug to a related given indication or therapeutic condition. The internal data source thus comprise structured data. The structured data may be logically linked or a relationship may be created with other data, such as reimbursement (e.g., INESSS, CADTH, Commercial Pipeline, etc.), data from other databases. The different links or relationships generally aim at providing a retrospective and prospective pipeline of clinical trial data.

The processing module 30 may further comprise a data governance and integration subsystem 40 comprising modules managing data access 41, data management 42, security of data 43 and operations on data 44. The data is feed to a natural language processing unit 45, a machine learning unit 46 and/or a deep learning unit 47. The natural language processing unit 45, a machine learning unit 46 and/or a deep learning unit 47 are configured to be trained using the data acquired from the different data sources 11 and classified in the repositories 31.

In some embodiments, the system 100 is configured to organize and structure at least some of the unstructured data sources, such as using Natural Language Processing (NLP) techniques.

The system further comprises an application module 50. The application module 50 is configured to execute instruction implementing the algorithms and to produce various analyses. In such an embodiment, a user of the system 100 will be able to obtain the following knowledge prediction of clinical phase transitions (Phase I, Phase II, Phase III and regulatory approval/pharmaco-economic approval), estimation of commercial success, prescriptive data for the optimization of study conduct (Classification of risk factors, recommendations for study design, estimation of recruitment rates etc.), better control of risk factors/events in the conduct of clinical trials through real-time monitoring of clinical study progress, more efficient use of internal resources (e.g., proactive classification of risk factors for each project by AI would allow for effective prioritization of clinical trials). More efficient use of internal resources (e.g., proactive classification of risk factors for each project by AI would allow for efficient prioritization of resources per project and optimize ROI), proactive management of clinical trial conduct (e.g., anticipation of recruitment needs, anticipation of impacting events such as adherence or drop-out), diligent analysis to evaluate investment opportunities (i.e., Merger & Acquisition, Venture Capital Investments).

Still referring to FIG. 3, the applications 50 are configured to use the results of the processed data to experiment, test and fine tune the said data. The output of the said experimentations, testing and tuning are deployed to predict, recommend, optimize, analyse, discover and/or report based on the processed data and the artificial intelligence engines (45, 46 and 47).

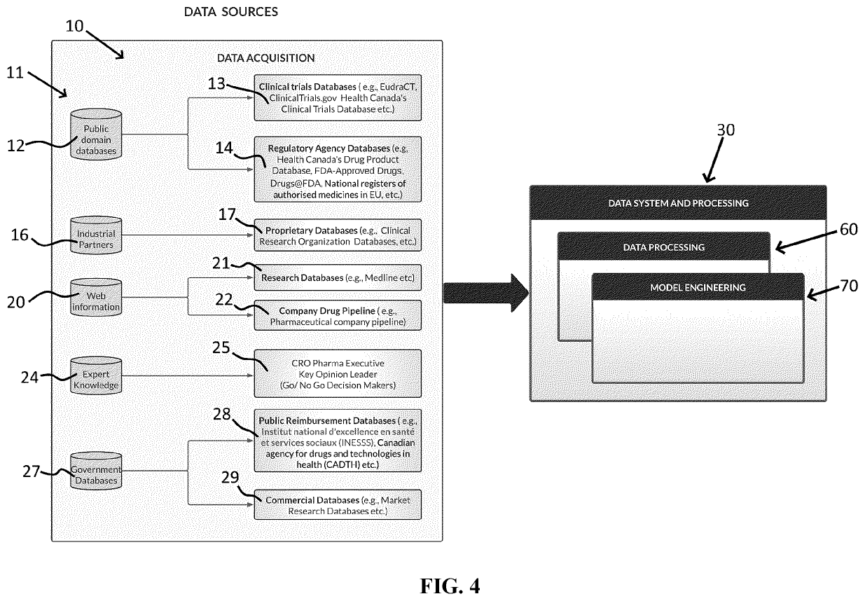

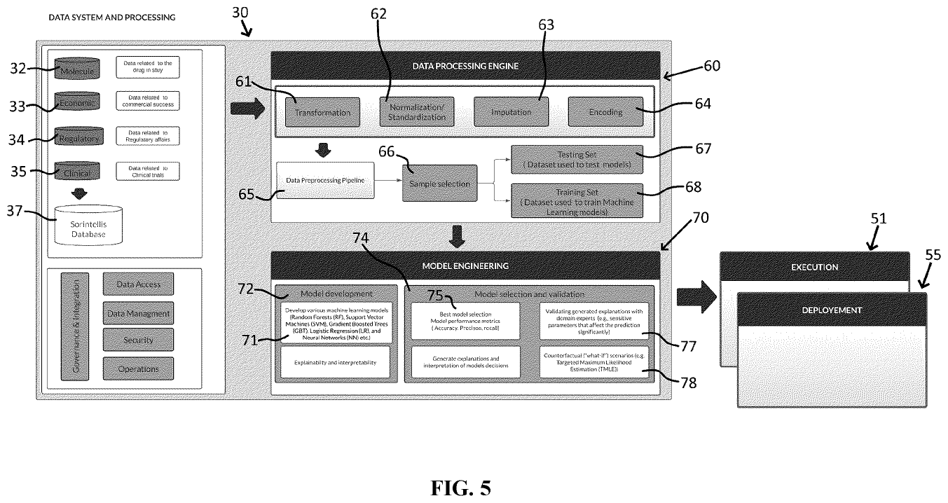

Referring to FIGS. 4 and 5, the application 50 may also be divided into a data processing engine 60 and a model engineering 70. The information collected in the data acquisition module 10 is sent to the data processing engine 60 for classifying, processing and/or standardizing the data. The data processing engine 60 may comprise a transformation module 61 configured to transform the data into an acceptable format, a normalization/standardization module 62, an imputation module 63 and an encoding module 64. The imputation module 63 may allow the treatment of missing data and incomplete data sets and the reduction of bias due to missing data. The encoding module 64 may allow the conversion of labelled data points into numerical variables for model training. Once the data is processed through one or more of the data processing modules 61, 62, 63 and/or 64, the said data is sent to data preprocessing pipeline 65. The data preprocessing pipeline 65 sends the processed data to a selection sampler 66. The selection sampler 66 is configured to divide the data into a testing 67 and training 68 sets. The testing set 67 is configured to test different models and the training set 68 is configured to train the machine learning unit 46.

The model engineering 70 is configured to receive the testing set 67 and/or the training set 68 as a basis for developing a plurality of models 71 and to select and validate the developed models 75. The model development module 72 is configured to develop machine learning models such as, but not limited to, Random Forests, Support Vector Machines, Gradient Boosted Trees, Logistic Regression, and Neural Networks, etc. The model development module 72 may further be configured to explain and interpret the developed models.

The model selection and validation module 74 may be used to select the best model developed in the model development module 72. The model selection and validation module 64 may thus comprise selecting the best model 75 based on performance metrics, such as the accuracy, the precision and the recall performance metrics. The model selection and validation module 74 may further comprise generating explanations and interpretation 76 of the decision used in the said model. The model selection and validation module 74 may further comprise validating the generated explanations and interpretations 77 with domain experts. As an example, the validation may comprise examining sensitive parameters that affect the predictions significantly. The model selection and validation module 74 may also comprise finding counterfactual scenarios 78. In some embodiments, the finding of counterfactual scenarios may comprise Targeted Maximum Likelihood Estimation models.

The selected models are then executed and deployed in production.

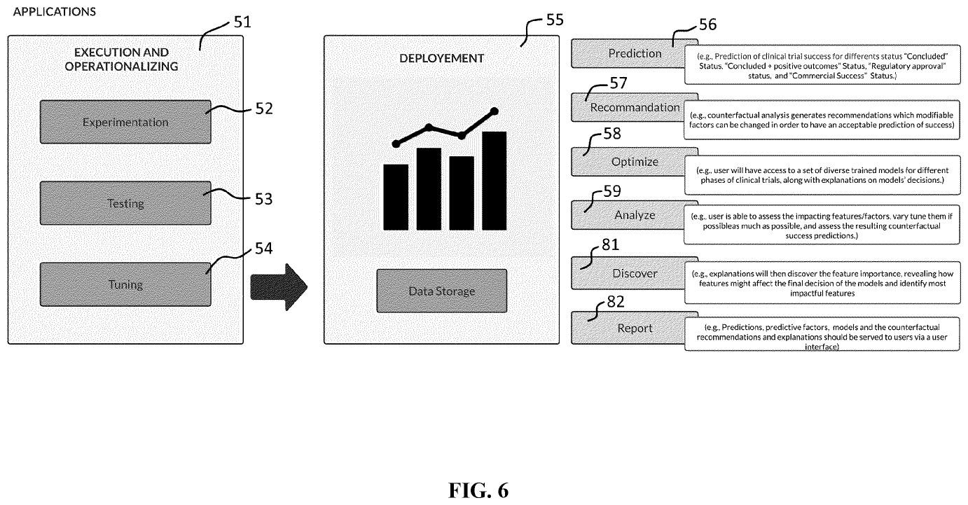

Referring now to FIGS. 5 and 6, once the model is validated by the model selection and validation module 74, the validated model is sent to the application module 50. The application module may comprise execution 51 and deployment 55. The execution 51 comprises an experimentation component 52, a testing component 53 and a tuning component 54. The execution module 51 thus allows to experiment, test, and fine-tune the model based on expectations and counterfactual scenarios. The deployment module 55 may use the chosen trained and tested model to make decisions based on new data. The deployment module 55 may thus comprise a prediction component 56 which may predict the clinical trial success for different statuses such as “concluded”, “concluded+positive outcomes”, “regulatory approval”, “commercial success”. The deployment module 55 may also comprise a recommendation component 57, which may generate recommendations on which modifiable factor may be changed in order to influence de predictions of the prediction component 56. The deployment module 55 may further comprise an optimization component 58, allowing the user to have access to a set of diverse trained models for different phases of clinical trials while providing the user with explanations on models' decisions. The deployment module 55 may further comprise an analyze component 59, allowing the user to assess the impacting features, vary these features and tune them and assess the counterfactual success predictions. The deployment module 55 may further comprise a discover component 81, allowing the user to discover the importance of certain features and reveal the impact of particular features on the final decisions. The deployment module 55 may further comprise a report component 82 configured to display on a user interface a summary of the predictions, the predictive factors, the models and the counterfactual recommendations and explanations.

In embodiments having a platform, the platform may be built to integrate a data environment organized into major clusters. In such an embodiment, each cluster represents a module: a clinical risk module, regulatory risk module, a commercial risk module and a pharmacological risk module. Each module comprises data that typically originates from public or private databases. The data is varied in nature, containing information on clinical trials, regulatory approval decisions, economic and reimbursement data, pharmacological data, commercial data and corporate data.

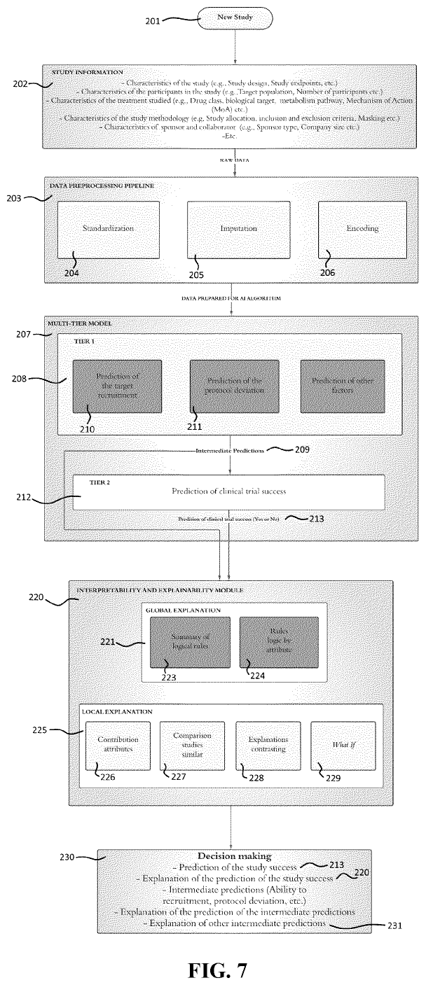

Referring to FIG. 7, a flowchart of an embodiment of a method for pharmaceutical portfolio strategic management decision support based on artificial intelligence 200 is shown. The method 200 comprises a receiving a request for a new treatment study 201. The request for a new treatment study 201 may comprise inputting one or more characteristics relating to the study 202. The characteristics relating to the study may comprise, but not limited to, the characteristics of the study, the characteristics of the participants in the study, the characteristics of the treatment to be studied, the characteristics of the methodology for treatment study, the characteristics of the sponsors and collaborators of the study, etc. The inputted information generally aims at collecting raw data about the study.

The inputted data, also referred to as raw data is inputted in a data preprocessing pipeline 203. The inputted data is standardized 204, imputed 205 and/or encoded 206. As such, the data is now formatted or processed to be inputted in an artificial intelligence (AI) program configured to execute an AI algorithm.

The preprocessed data is inputted in a multi-tier model 207 to calculate predictions about the clinical trial success of the submitted study. As an example, the tier one model 208 may be configured to calculate intermediate predictions 209 of some aspects of the clinical trial success. As examples, the tier one model 208 may comprise calculating predictions on the target recruitment 210, calculating prediction of the protocol deviation 211, and calculating prediction of other factors. The predictions according to the different aspects represent intermediate predictions of the success of the clinical study.

As an example, the tier one model 208, being the lowest level in the exemplary structure, the prediction target may correspond to the business needs of a contract research organization (CRO), specifically predicting whether the clinical trial will be completed or not. The method may thus comprise developing a machine learning model (tier 1) for predicting the success based on the specific business needs of a CRO. The developed model identifies among all the factors the ones that have a greater impact on the resulting prediction of the developed model. The identified factors may include but are not limited to protocol deviation, number of recruited patients, duration of the clinical trial, dropout rate, and/or age of patients in the trial. The identified factors may be the most significant predictive contributors. The most significant predictive contributors, unlike hundreds of other variables in the database, are unknown at the beginning of the clinical trial. As a result, such variables may be used in tier one model 208 to predict the success of the clinical trial.

The intermediate predictions 209 are inputted in a tier two model 212. The tier two model 212 is configured to make a prediction regarding the clinical trial success 213 based on the intermediate predictions 209 of a tier one model 208. Understandably, the intermediate predictions 209 of the most contributing variables, combined, shall effectively predict the target of interest (completion or non-completion of the clinical trial). In the illustrated workflow, in tier one 208 the model is expected to evolve with the prediction of subsequent targets until commercial success.

The calculated intermediate predictions 209 and the calculated prediction of success of the clinical trial 213 are inputted in an interpretability and explainability module 220. The interpretability and explainability module 220 generally aims at interpreting and explaining the calculated intermediate predictions 209 and the calculated prediction of success of the clinical trial 213. At each stage of the process, the method provides an intermediate prediction 208. The intermediate prediction 209 identifies the level of impact of the variables being used such model 208. Understandably, the level of impact ranges across positive, neutral or negative impacts. As such, as the multi-tier model 207 may comprise a plurality of tiers, each tier identifies levels of impact of each variable being used in each model of the tier. As such, the system 100 comprises explanations of the predicted success of the clinical study by analysing the intermediate predictions and the different impacts of the variables of such models of the tiers. The different intermediate predictions thus reveals an interpretation of these variables at each stage of the multi-tier model 207 to provide explainability and interpretability.

In some embodiments, the module 220 may comprise a global explanation subsystem 221 configured to generate a summary of logical rules 223 and rules logic by attribute 224. and the module may further comprise a local explanation module 225 configured to generate or produce the contribution attributes 226, the comparison of or to similar studies 227, the contrasting explanations 228 and the counterfactual (What if) scenarios 229. As explained above, the global explanation subsystem 221 and local explanation module 225 use the intermediate predictions 208 and the relevant variables outputted at each stage of the multi-tier 207 model to generate logical rules or other contribution attributes. The interpretability and explainability module 220 may be configured to generate and present explanations or basis of the predictions made by the AI algorithm to users. As such, the users may make better and informed decisions concerning the study at hand.

The method 200 further comprises making a decision 230. The decision making 230 may comprise using the prediction of the study success 213, the explanation of the predictions 220, the intermediate predictions 208, the explanations of the intermediate predictions and of other predictions 231.

The use of a multi-stage model generally aims at breaking down complex decision-making process into a plurality of stages, tiers or phases. Furthermore, the use of a multi-stage model 207 discloses the specific impact of each of the tiers and the associated variables over the resulting prediction of success of the clinical trial. In such embodiment, each stage or tier represents a specific aspect or consideration in the decision-making process and one or more predictions are calculated for each the stages. The multi-stage approach generally aims at providing a systematic and comprehensive analysis. The predictions of each stage or tier is typically used as an input source for the next stage or tier. As such, the next stage uses the result from the previous stage to calculate another prediction, which is enhanced in relation to the intermediate predictions. As such, the system may break down the complex problem of predicting success or no success of a clinical trial study into plurality of calculation of predictions for smaller problematics. The use of multistage model further allow improving overall performance of the engine.

In embodiments using a multistage model, the method 200 generally aims at unlocking or calculating predictions for specific situations or aspects of the global prediction by providing a more targeted and modular approach to problem-solving. By breaking down a complex problem into simpler steps, each intermediate model can focus on a specific aspect and solve particular sub-problems. Interpreting the results and understanding the information provided by these models or stages is crucial to comprehend which issue is being resolved and utilize the information provided by the models effectively. For example, multistage models may be used in predicting the percentage chance of success in achieving the target number of participants for a phase 2 clinical study, or assessing the likelihood of a phase 2 clinical study belonging to a high-risk zone for protocol deviation, or estimating the duration, in days, of a phase 2 clinical study.

Each stage of a multistage model generally requires a separate training and evaluation process, as well as the management of intermediate inputs and outputs. For example, the multistage model may comprise a stage linked to the estimation of volunteer recruitment for a clinical study, a stage linked to the estimation of protocol deviation, a stage linked to the estimation of volunteer withdrawal from a study, or a stage linked to the estimation of study duration. Understandably, any other number of aspects or stages may be contemplated within the scope of the present invention.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

### REFERENCES

CenterWatch. (n.d.). Retrieved Jun. 2, 2022, from https://www.centerwatch.com/articles/16879
Babbs, C. F. (2014). Choosing inclusion criteria that minimize the time and cost of clinical trials. World Journal of Methodology, 4 (2), 109-122. https://doi.org/10.5662/wjm.v4.i2.109
Dhankhar, A., Saptarshi, G., Arvind, G., & Michael, T. (2018). Expanding horizons for risk management in pharma|Mckinsey. https://www.mckinsey.com/business-functions/risk-and-resilience/our-insights/expanding-horizons-for-risk-management-in-pharma
Dickson, M., & Gagnon, J. P. (2004). Key factors in the rising cost of new drug discovery and development. Nature Reviews. Drug Discovery, 3 (5), 417-429. https://doi.org/10.1038/nrd1382
Fogel, D. B. (2018). Factors associated with clinical trials that fail and opportunities for improving the likelihood of success: A review. Contemporary Clinical Trials Communications, 11, 156-164. https://doi.org/10.1016/j.conctc.2018.08.001
Fouad, M. N., Lee, J. Y., Catalano, P. J., Vogt, T. M., Zafar, S. Y., West, D. W., Simon, C., Klabunde, C. N., Kahn, K. L., Weeks, J. C., & Kiefe, C. I. (2013). Enrollment of patients with lung and colorectal cancers onto clinical trials. Journal of Oncology Practice, 9 (2), e40-47. https://doi.org/10.1200/JOP.2012.000598
Getz, K. A., Zuckerman, R., Cropp, A. B., Hindle, A. L., Krauss, R., & Kaitin, K. I. (2011). Measuring the Incidence, Causes, and Repercussions of Protocol Amendments. Drug Information Journal: DIJ/Drug Information Association, 45 (3), 265-275. https://doi.org/10.1177/009286151104500307
Harrison, R. K. (2016). Phase II and phase III failures: 2013-2015. Nature Reviews. Drug Discovery, 15 (12), 817-818. https://doi.org/10.1038/nrd.2016.184
Hoskyn, S. L. (n.d.). EXPLAINING PUBLIC REIMBURSEMENT DELAYS FOR NEW MEDICINES FOR CANADIAN PATIENTS. 3.
Hwang, T. J., Carpenter, D., Lauffenburger, J. C., Wang, B., Franklin, J. M., & Kesselheim, A. S. (2016). Failure of Investigational Drugs in Late-Stage Clinical Development and Publication of Trial Results. JAMA Internal Medicine, 176 (12), 1826-1833. https://doi.org/10.1001/jamainternmed.2016.6008
Kwak, Y., & Dixon, C. (2008). Risk management framework for pharmaceutical research and development projects. https://doi.org/10.1108/17538370810906255
Lamberti, M. J., Mathias, A., Myles, J. E., Howe, D., & Getz, K. (2012). Evaluating the Impact of Patient Recruitment and Retention Practices. Drug Information Journal: DIJ/Drug Information Association, 46 (5), 573-580. https://doi.org/10.1177/0092861512453040
Pammolli, F., Magazzini, L., & Riccaboni, M. (2011). The productivity crisis in pharmaceutical R&D. Nature Reviews. Drug Discovery, 10 (6), 428-438. https://doi.org/10.1038/nrd3405
Paul, S. M., Mytelka, D. S., Dunwiddie, C. T., Persinger, C. C., Munos, B. H., Lindborg, S. R., & Schacht, A. L. (2010a). How to improve R&D productivity: The pharmaceutical industry's grand challenge. Nature Reviews Drug Discovery, 9 (3), 203-214. https://doi.org/10.1038/nrd3078
Paul, S. M., Mytelka, D. S., Dunwiddie, C. T., Persinger, C. C., Munos, B. H., Lindborg, S. R., & Schacht, A. L. (2010b). How to improve R&D productivity: The pharmaceutical industry's grand challenge. Nature Reviews. Drug Discovery, 9 (3), 203-214. https://doi.org/10.1038/nrd3078
PMBOK® Guide. (n.d.). Retrieved Jun. 14, 2022, from https://www.pmi.org/pmbok-guide-standards/foundational/PMBOK
Scannell, J. W., Blanckley, A., Boldon, H., & Warrington, B. (2012). Diagnosing the decline in pharmaceutical R&D efficiency. Nature Reviews. Drug Discovery, 11 (3), 191-200. https://doi.org/10.1038/nrd3681
Schlander, M., Hernandez-Villafuerte, K., Cheng, C.-Y., Mestre-Ferrandiz, J., & Baumann, M. (2021). How Much Does It Cost to Research and Develop a New Drug? A Systematic Review and Assessment. PharmacoEconomics, 39 (11), 1243-1269. https://doi.org/10.1007/s40273-021-01065-y
Schroen, A. T., Petroni, G. R., Wang, H., Gray, R., Wang, X. F., Cronin, W., Sargent, D. J., Benedetti, J., Wickerham, D. L., Djulbegovic, B., & Slingluff, C. L. (2010). Preliminary evaluation of factors associated with premature trial closure and feasibility of accrual benchmarks in phase III oncology trials. Clinical Trials (London, England), 7 (4), 312-321. https://doi.org/10.1177/1740774510374973
Schuhmacher, A., Gassmann, O., & Hinder, M. (2016). Changing R&D models in research-based pharmaceutical companies. Journal of Translational Medicine, 14 (1), 105. https://doi.org/10.1186/s12967-016-0838-4
Serebruany, V. L., Oshrine, B. R., Malinin, A. I., Atar, D., Michelson, A. D., & Ferguson, J. J. (2005). Noncompliance in cardiovascular clinical trials. American Heart Journal, 150 (5), 882-886. https://doi.org/10.1016/j.ahj.2005.02.039
Smith, D. L. (2012). Patient Nonadherence in Clinical Trials: Could There Be a Link to Postmarketing Patient Safety? Drug Information Journal: DIJ/Drug Information Association, 46 (1), 27-34. https://doi.org/10.1177/0092861511428300
Thoma, A., Farrokhyar, F., McKnight, L., & Bhandari, M. (2010). How to optimize patient recruitment. Canadian Journal of Surgery, 53 (3), 205-210. https://www.ncbi.nlm.nih.gov/pmc/articles/PMC2878987/
Williams, R. J., Tse, T., DiPiazza, K., & Zarin, D. A. (2015). Terminated Trials in the ClinicalTrials.gov Results Database: Evaluation of Availability of Primary Outcome Data and Reasons for Termination. PloS One, 10 (5), e0127242. https://doi.org/10.1371/journal.pone.0127242

1. A system for predicting level of success of a clinical trial of a pharmacological product comprising:
a data source comprising data relating to the clinical trial;
a server comprising:
a data acquisition module in data communication with a plurality of external data sources comprising data relating to clinical trials, regulatory approvals, economic and reimbursement information, pharmacological information, and commercial and corporate information;
a data processing engine configured to transform and normalize data from the data acquisition module using a natural language processor;
a machine learning engine comprising a model trained with the data processed by the data processing engine, the trained machine learning engine being configured to execute an algorithm to analyse the data of the data source relating to the clinical trial and to calculate a prediction of success of the clinical trial based on the said analyzed data.

2. The system of claim 1, the model being trained by the machine learning engine being a multi-tiers model.

3. The system of claim 2, the multi-tiers model comprising first-tier model configured to calculate a plurality of intermediate predictions of success.

4. The system of claim 3, the multi-tiers model comprising a plurality of first tier models, each model being configured to calculate an intermediate prediction of success.

5. The system of claim 4, the plurality of first tier models comprising at least one of the following models:
a model to calculate prediction of target recruitment of the clinical trial;
a model to calculate a prediction of protocol deviation of the clinical trial; and
a model to calculate other factors relating to the clinical trials.

6. The system of claim 4, the intermediate prediction of success of each of the first-tier models being inputted in second-tier model to calculate the prediction of the success of the clinical trial.

7. The system of claim 1 further comprising a module to interpret and explain the calculated prediction of success of the clinical trial.

8. The system of claim 7, the module to interpret and explain the calculated prediction of success of the clinical trial comprising generating logical rules used to calculate the prediction.

9. The system of claim 7, the module to interpret and explain the calculated prediction of success of the clinical trial comprising any of the followings:
contribution attributes of the clinical trials;
studies used to compare to the clinical trial;
contrasting explanations;
scenarios impacting level of predicted success of the clinical trial.

10. The system of claim 1 further comprising an application module configured to execute the machine learning engine with data relating to the clinical trial.

11. The system of claim 1, the acquired data source being classified in plurality of repositories.

12. The system of claim 11, the repositories comprising any one of the following type of data:
clinical data, regulatory data, economic data, molecule data and MPP.

13. A computer-implemented method for predicting level of success of a clinical trial of a pharmacological product comprising:
acquiring data from a plurality of external data sources comprising data relating to clinical trials, regulatory approvals, economic and reimbursement information, pharmacological information, and commercial and corporate information;
processing, transforming and normalizing the acquired data using a natural language processor,
executing a machine learning model trained with the processed, transformed and normalized data to analyse data relating to the clinical trial and to calculate a prediction of success of the clinical trial based on the said analyzed data.

14. The method of claim 13, the trained model being a multi-tiers model comprising a plurality of first-tier models and a second-tier model, the method further comprising each of the first-tier model calculating an intermediate prediction of success of a specific aspect of the clinical study.

15. The method of claim 14, each of the plurality of first tier models calculating one of the followings:
a prediction of target recruitment of the clinical trial;
a prediction of protocol deviation of the clinical trial; and
other factors relating to the clinical trials.

16. The method of claim 15, the second-tier model using each of the intermediate predications calculated by the first-tier models to calculate the prediction of success of the clinical trial.

17. The method of claim 13 further comprising monitoring in real-time progress characteristics of the clinical study using such characteristics to calculate the prediction of success of the clinical trial.

18. The method of claim 17, the characteristics comprising anticipation of recruitment needs and identification of impacting events.

19. The method of claim 13, the execution of the machine learning model further calculating any one of the followings: clinical risk of the clinical trial, regulatory risk of the clinical trial, commercial risk of the clinical trial and pharmacological risk of the clinical trial.

20. The method of claim 13, the execution of the machine learning model further generating prescriptive data for optimizing study conduct.

21. The method of claim 13 further comprising developing a plurality of machine learning model for the clinical trial, training the developed models with acquired data and selecting one or more of the developed models based on performance metrics.

22. A computer-readable medium storing instructions for executing the method of claim 13.
